(19) United States
(12) Patent Application Publication
(10) Pub. No.: US 2016/0081759 A1
(43) Pub. Date: Mar. 24, 2016

(54) METHOD AND DEVICE FOR STEREOSCOPIC DEPICTION OF IMAGE DATA

(71) Applicant: SIEMENS AKTIENGESELLSCHAFT, München (DE)

(72) Inventor: Anton SCHICK, Velden (DE)

(73) Assignee: SIEMENS AKTIENGESELLSCHAFT, München (DE)

(21) Appl. No.: 14/785,289

(22) PCT Filed: Apr. 10, 2014
(86) PCT No.: PCT/EP2014/057231

§ 371 Date: Oct. 16, 2015

(30) Foreign Application Priority Data
Apr. 17, 2013 (DE) 102013206911.1

(51) Int. Cl. A61B 19/00; G06T 7/00; G06T 15/04; G06T 11/60; A61B 1/00; A61B 17/00

(52) U.S. Cl. CPC A61B 19/5244; A61B 1/0005; A61B 1/00193; A61B 17/00234; A61B 1/00009; G06T 15/04; G06T 11/60; G06T 7/0059; A61B 2019/5236; A61B 2019/524; A61B 2019/5276; A61B 2560/0487; A61B 2019/5295; A61B 2019/5257; A61B 2017/00207; G06T 2207/10028; G06T 2207/10072; G06T 2207/20221

(57) ABSTRACT

Three-dimensional depiction of image information is provided during minimally invasive surgery carried out by an endoscope by detecting an operation region of the using a sensor device in three dimensions. Stereoscopic image data is generated from the 3D-data acquired from the sensor and is visualized on a suitable display device.

### CROSS REFERENCE TO RELATED APPLICATIONS

This application is the U.S. national stage of International Application No. PCT/EP2014/057231, filed Apr. 10, 2014 and claims the benefit thereof. The International Application claims the benefit of German Application No. 10201306911.1 filed on Apr. 17, 2013, both applications are incorporated by reference herein in their entirety.

### BACKGROUND

Described below are a method and a device for the stereoscopic depiction of image data, in particular a method and a device for the stereoscopic depiction of image data in minimally invasive surgery.

Endoscopic treatments and examinations in the field of medicine enable significantly more gentle and less traumatic treatment in comparison to an open intervention on the patient. Therefore, these treatment methods are gaining increasingly large significance. During a minimally invasive intervention, optical and surgical instruments (endoscopes) are introduced into the body of a patient by an operator via one or more relatively small accesses on the body of the patient. The operator can therefore carry out an examination and treatment using the surgical instruments. At the same time, this procedure can be monitored through the optical instruments. Simple endoscopes enable in this case either a direct view through an eyepiece of the endoscope or an observation of the region to be operated on via a camera attached to the endoscope and an external monitor. Three-dimensional vision is not possible in the case of this simple endoscope. If the endoscope additionally has a second observation channel, which enables an observation of the object from a second direction, three-dimensional vision can thus be enabled, in that both directions are led outward by two eyepieces for the right and left eyes. Since, in the case of a single endoscope, the distance between the observation channels is generally very small (typically at most 6 mm), such a stereoscopic endoscope also only delivers very restricted three-dimensional vision in the microscopic range. For a three-dimensional observation which corresponds to a human eye spacing of approximately 10 cm, it is therefore necessary to provide an access channel which is spaced apart further. Since a further opening on the body of the patient for an additional access channel is linked to further traumatization of the patient, however, an additional access channel is to be avoided if possible.

If a three-dimensional visualization of the treatment region by way of a single endoscope is to be enabled in minimally invasive surgery, therefore either two observation beam paths have to be led outward inside the cross section of the endoscope, or alternatively two cameras spaced apart from one another have to be arranged on the endoscope tip, as was stated above. In both cases, because of the very limited cross section of the endoscope, only extremely low three-dimensional resolution is possible, which results in greatly restricted resolution of the depiction range.

Alternatively, it is also possible to survey the treatment region in the interior of the patient three-dimensionally by a digital system. Document DE 10 2006 017 003 A1 discloses, for example, an endoscope having optical depth data acquisition. For this purpose, modulated light is emitted into the treatment region and depth data of the treatment space are calculated based on the received light signal.

In this case, the direct three-dimensional view into the treatment region remains denied to the operator even after the ascertainment of the available depth data in the interior of the treatment space. The operator has to plan and execute his or her treatment based on a model depicted on a two-dimensional display screen.

A demand therefore exists for improved stereoscopic depiction of image data, in particular a demand exists for a stereoscopic depiction of image data in minimally invasive surgery.

The method for the stereoscopic depiction of image data in minimally invasive surgery includes at least partial three-dimensional acquisition of a surface; preparation of a depth map of the at least partially three-dimensionally acquired surface; texturing of the prepared depth map; calculation of stereoscopic image data from the textured depth map; and visualization of the calculated stereoscopic image data.

### SUMMARY

Described below is a device for the stereoscopic depiction of image data in minimally invasive surgery having a sensor device, which is designed to at least partially three-dimensionally acquire a surface; a device for preparing a depth map, which is designed to prepare a depth map from the at least partially three-dimensionally acquired surface; a texturing device, which is designed to texture the prepared depth map; an image data generator, which is designed to calculate stereoscopic image data from the textured depth map; and a visualization device, which is designed to visualize the calculated stereoscopic image data.

The method firstly three-dimensionally acquires a region which is not directly accessible by way of a sensor and prepares a digital model in the form of a depth map from this three-dimensional acquisition. Stereoscopic image data, which are optimally adapted to the eye spacing of the user, can then be generated automatically in a simple manner for a user from this depth map.

By three-dimensional surveying of the observation region using a special sensor system, the inaccessible region, for example, in the body interior of a patient, can be acquired in this case by a sensor having a very small structural size. The data thus acquired can be conducted outward in a simple manner, without an endoscope having a particularly large cross section being required for this purpose.

Therefore, outstanding spatial acquisition of the treatment region is achieved, without an endoscope having an extraordinarily large cross section or further accesses to the operation region in the body interior of the patient being required for this purpose.

A further advantage is that such a sensor system can acquire the region to be acquired in a very good three-dimensional resolution and a correspondingly high number of pixels, since the sensor on the endoscope only requires a single camera. Therefore, with only little traumatization of the patient, the operation region to be monitored can be depicted in a very good image quality.

A further advantage is that a stereoscopic visualization of the region to be monitored, which is optimally adapted to the eye spacing of a user, can be generated from the three-dimensional data provided by the sensor system. Therefore, the visualization of the image data can be prepared for a user so that optimum three-dimensional acquisition is possible.

It is furthermore advantageous that the calculation of the stereoscopic image data is performed independently of the three-dimensional acquisition of the object surface by the sensor. A stereoscopic depiction of the treatment region, which deviates from the present position of the endoscope, can therefore also be provided to a user.

By way of suitable preparation of the depth map from the three-dimensionally acquired object data, a depiction of the treatment region, which comes very close to the real conditions, can therefore be provided to a user.

According to one embodiment, the calculated stereoscopic image data correspond to two viewing directions of two eyes of a user. By way of the preparation of the stereoscopic image data in accordance with the viewing directions of the eyes of the user, an optimum stereoscopic visualization of the treatment region can be enabled for the user.

In one embodiment, the depth map includes spatial points of the at least partially three-dimensionally acquired surface. Such a depth map enables very good further processing of the three-dimensionally acquired surface.

According to one embodiment, the three-dimensional acquisition of the surface is executed continuously, and the depth map is adapted based on the continuously three-dimensionally acquired surface. In this manner, it is possible to supplement and if necessary also correct the depth map continuously, so that a complete three-dimensional model of the region to be observed is successively constructed. Therefore, after some time, image information can also be provided about regions which initially could not be acquired because of shadows or similar effects.

In a further embodiment, the method includes providing further items of image information and combining the further items of image information with the acquired three-dimensional surface. By way of this combination of the three-dimensionally acquired surface with further image data, a particularly good and realistic visualization of the stereoscopic image data can be enabled.

In a special embodiment, the further items of image information are diagnostic image data, in particular data from computer tomography, magnetic resonance tomography, an x-ray picture, and/or sonography. Such diagnostic image data, which were prepared before or during the treatment and are related to the treatment region to be observed, provide particularly valuable items of information for the preparation and visualization of the treatment region. For example, these image data can be provided directly by the imaging diagnostic devices, or a storage device 21.

In a further embodiment, the image data are calculated for a predefined viewing direction in calculating the stereoscopic image data. This viewing direction can be different from the present position of the endoscope having the sensor for three-dimensional acquisition of the surface. A particularly flexible visualization of the treatment region can thus be achieved.

In a special embodiment, the method includes acquiring a user input, wherein the predefined viewing direction is adapted in accordance with the acquired user input. It is therefore possible for the user to adapt the viewing direction individually to his or her needs.

In a further embodiment of the device, the sensor device is arranged on or in an endoscope.

In a special embodiment, the endoscope furthermore includes at least one surgical instrument. It is therefore possible simultaneously to execute a surgical intervention and visually monitor this intervention at the same time through a single access.

In one embodiment, the device includes a sensor device having a time-of-flight camera and/or a device for triangulation, in particular a device for active tri-angulation. A particularly good three-dimensional acquisition of the surface can be achieved by such sensor devices.

In a further embodiment, the sensor device includes a camera, which may be a color camera. In addition to the three-dimensional acquisition of the surface, digital image data, which are used for the visualization of the treatment region, can thus also be obtained simultaneously by the sensor device.

In a further embodiment, the image data generator calculates the image data for a predefined viewing direction.

In a special embodiment, the device includes an input device, which is designed to acquire an input of a user, wherein the image data generator calculates the stereoscopic image data for a viewing direction based on the input of the user.

In a further special embodiment, the input device acquires a movement of the user in this case, in particular a gesture performed by the user. This movement or gesture may be acquired by a camera.

### BRIEF DESCRIPTION OF THE DRAWINGS

These and other features and advantages will become more apparent and more readily appreciated from the following description of the exemplary embodiments with reference to the accompanying drawings of which:

FIG. 1 is a schematic block diagram of a device for the stereoscopic depiction of image data of a body illustrated in cross section, according to one embodiment;

FIG. 2 is a schematic block diagram of the components of a device according to a further embodiment;

FIGS. 3 and 4 are schematic perspective views of monitor elements for a stereoscopic visualization; and

FIG. 5 is a flowchart of a method for the stereoscopic depiction of image data, on which a further embodiment is based.

### DETAILED DESCRIPTION OF THE PREFERRED EMBODIMENT

Reference will now be made in detail to the preferred embodiments, examples of which are illustrated in the accompanying drawings, wherein like reference numerals refer to like elements throughout.

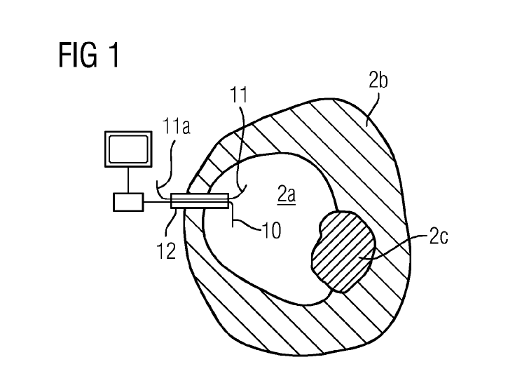

FIG. 1 shows a schematic illustration of a minimally invasive intervention using an endoscope, which includes a device for stereoscopic depiction according to one embodiment. In the body 2 of a patient, an endoscope 12 is introduced into the body 2b in this case via an access 2d. In this case, the treatment space 2a can be widened, for example, by introducing a suitable gas, after the access 2d has been sealed accordingly. A sufficiently large treatment space therefore results in front of the treated object 2c. In the treatment space 2a, a sensor device 10, on the one hand, and additionally one or more surgical instruments 11 can be introduced into the treatment space by way of the endoscope 12. The surgical instruments 11 can be controlled from the outside in this case by a suitable device 11a, to carry out the treatment in the interior 2a.

The visual monitoring of this treatment is performed in this case by the sensor device 10. This sensor device 10 is a sensor in this case, which can three-dimensionally acquire the surface of the treatment space 2a and at the same time in particular also the surface of the treated object 2c. The sensor device 10 can be a sensor, for example, which operates according to the principle of a time-of-flight camera (ToF camera). In this case, modulated light pulses are emitted from a light source and the light which is scattered and reflected from the surface is analyzed by an appropriate sensor, for example a camera. A three-dimensional model can then be prepared based on the propagation speed of the light.

Alternatively, for example, the sensor device 10 can also carry out a triangulation to ascertain the three-dimensional location of the surface in the treatment space 2a. Fundamentally, such a triangulation can be performed, for example, by passive triangulation using two separate cameras. However, since solving the correspondence problem is difficult in the case of passive triangulation with low-contrast surfaces (for example, the liver) and the 3D data density is very low, active triangulation is preferably performed. In this case, a known pattern is projected onto the surface in the treatment space 2a by the sensor device 10 and the surface is recorded at the same time by a camera. The projection of the known pattern on the surface may be performed using visible light. Additionally or alternatively, however, the operation region can also be illuminated using light outside the visible wavelength range, for example using infrared or ultraviolet light.

By way of a comparison of the pattern recorded by the camera on the surface of the treatment space 2a with the known ideal pattern emitted from the projector, the surface of the treatment space 2a can thereupon be three-dimensionally acquired and analyzed.

In this case, the treatment space 2a and its surface can also be acquired by the camera, simultaneously or alternatively to the three-dimensional acquisition of the surface. In this manner, a corresponding color or black-and-white image of the treatment space 2a can be acquired. In this case, the light sources of the sensor device 10 can also be used simultaneously for illuminating the treatment space 2a, to obtain image data.

The data acquired by the sensor device 10 about the three-dimensional location of the surface in the treatment space 2a, and also the color or black-and-white image data acquired by the camera, are fed outward and are therefore available for further processing, in particular visualization.

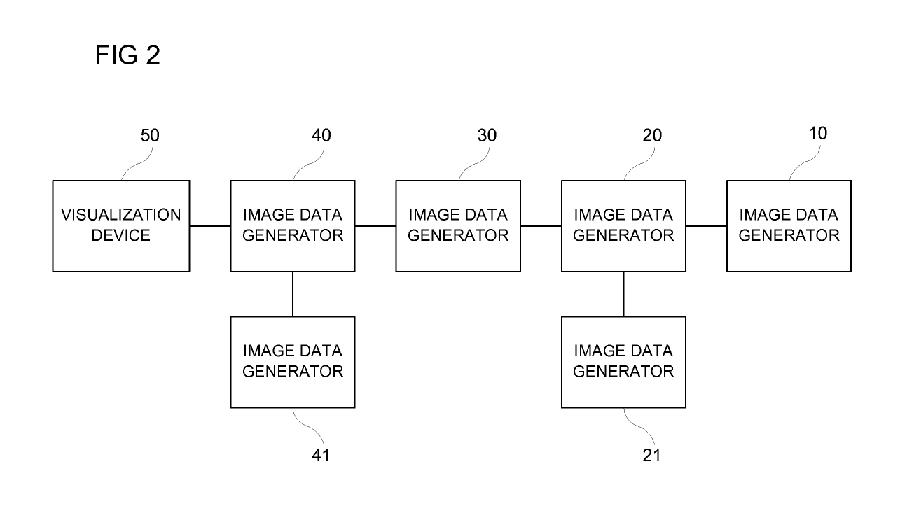

FIG. 2 shows a schematic illustration of a device for the visualization of stereoscopic image data, as have been generated, for example, from the example described in conjunction with FIG. 1. The sensor device 10 acquires in this case a surface located in the field of vision of the sensor device 10 and its three-dimensional location of individual surface points in space. As described above, in this case, additionally or alternatively to the three-dimensional acquisition of the spatial points, a known acquisition of image data can be performed by a black-and-white or color camera. The information about the three-dimensional location of the spatial points is then supplied to a device 20 for preparing a depth map. This device 20 for preparing a depth map analyzes the items of information about the three-dimensional location of the surface points from the sensor device 10 and generates a depth map therefrom, which includes information about the three-dimensional location of the spatial points acquired by the sensor device 10.

Since the sensor device 10 only has a restricted field of vision and additionally some partial regions also initially cannot be acquired because of shadows, for example, as a result of protrusions in the treatment space 2a, at the beginning of the three-dimensional acquisition of the surface in the treatment space 2a, the depth map will initially have larger or smaller gaps. By way of further continuous acquisition of the surface in the treatment space 2a by the sensor device 10, the prepared depth map will be completed more and more in the course of time and in particular if the sensor device 10 moves inside the treatment space 2a. Therefore, items of information about spatial points which presently cannot be acquired by the sensor device 10 because they are located outside the field of vision or behind a shadow, for example, are also provided in this depth map after some time. In addition, by way of the continuous acquisition of the surface by the sensor device 10, a change of the surface can also be corrected in the depth map. The depth map therefore always reflects the presently existing state of the surface in the treatment space 2a.

The spatial points of the surface of the treatment space 2a present in the depth map are relayed to a texturing device 30. The texturing device 30 can optionally combine the items of information from the depth map in this case with the image data of an endoscopic black-and-white or color camera. The texturing device 30 generates a three-dimensional object having a coherent surface from the spatial points of the depth map. In this case, the surface can be suitably colored or shaded as needed by combining the three-dimensional spatial data of the depth map with the endoscopic camera data.

Furthermore, it is additionally possible to also incorporate additional diagnostic image data. For example, recordings can already be prepared of the treated region preoperatively. Imaging diagnostic methods are suitable for this purpose, for example, computer tomography (CT), magnetic resonance tomography (MR or MRT), x-ray pictures, sonography, or similar methods. It is also conceivable to generate additional items of information, which can also be incorporated into the image generation process, if necessary during the treatment by way of suitable imaging diagnostic methods.

After texturing of the surface of the treatment space 2a has been carried out from the image data of the depth map and optionally the further image data in the texturing device 30, the items of information thus processed are relayed to an image data generator 40. This image data generator 40 generates stereoscopic image data from the items of textured three-dimensional information. These stereoscopic image data include at least two images, which are slightly offset in relation to one another, and which take into consideration the eye spacing of a human observer. The spacing used between the two eyes is typically approximately 80 mm in this case. The user receives a particularly good three-dimensional impression in this case if it is presumed that the object to be observed is located approximately 25 cm in front of his or her eyes. Fundamentally, however, other parameters are also possible, which enable a three-dimensional impression of the object to be observed for an observer. The image data generator 40 therefore calculates at least two image data sets from a predefined viewing direction, wherein the viewing directions of the two image data sets differ by the eye spacing of an observer. The image data thus generated are subsequently supplied to a visualization device 50. If still further information or data are to be required for the visualization device 50 for a three-dimensional depiction, they can also be generated and provided by the image data generator 40.

In this case, all devices which are capable of providing different items of image information in each case to the two eyes of an observer are suitable as the visualization device 50. For example, the visualization device 50 can be a 3D monitor, or special spectacles, which display different image data for the two eyes of a user.

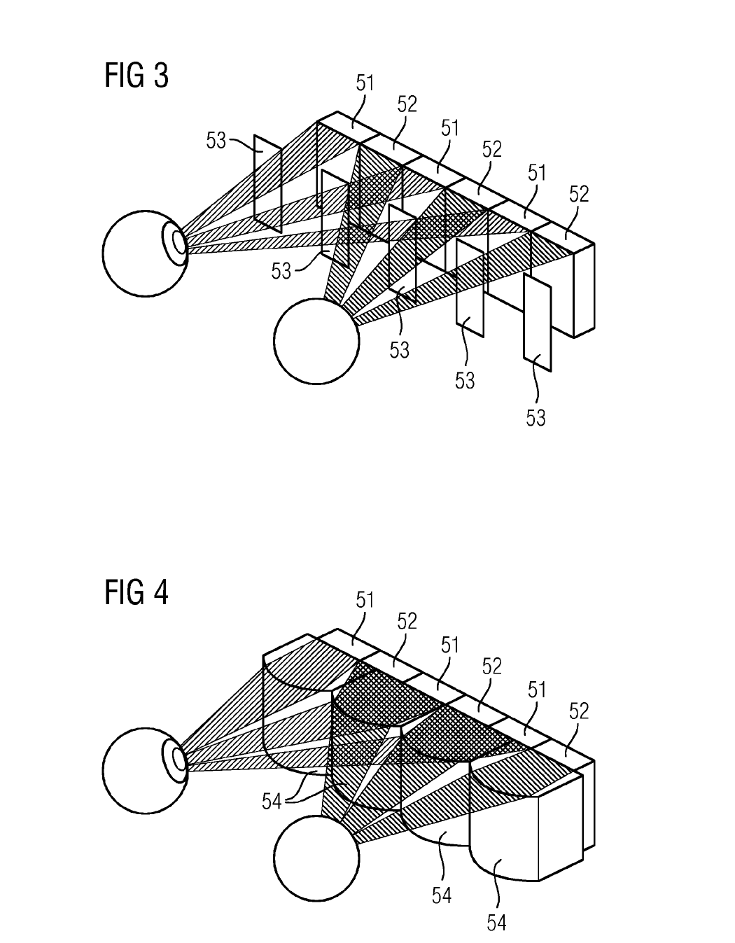

FIG. 3 shows a schematic illustration of a detail of pixels for a first embodiment of a 3D monitor. Pixels 51 for a left eye and pixels 52 for a right eye are arranged alternately adjacent to one another on the display screen in this case. Because of a slotted aperture 53 arranged in front of these pixels 51 and 52, the left and the right eye only see the respective pixels intended for them in this case, while the pixels for the respective other eye of the user are covered by the slotted aperture 53 as a result of the respective viewing direction.

FIG. 4 shows an alternative form of a 3D monitor. In this case, small lenses 54, which deflect the beam path for the left and the right eye so that each eye only sees the pixels intended for the corresponding eye, are arranged in each case in front of the pixels 51 for the left eye and the pixels 52 for the right eye.

Fundamentally, all other types of 3D capable monitors are additionally also conceivable and suitable. Thus, for example, monitors can also be used which emit light having a different polarization in each case for the left and the right eye. In this case, however, the user must wear spectacles having a suitable polarization filter. In the case of monitors which alternately output image data for the left and the right eye, a user also has to wear suitable shutter spectacles, which each only release the view on the monitor for the left and the right eye alternately in synchronization with the alternately displayed images. Because of the comfort losses which accompany wearing spectacles, however, visualization devices which operate according to the principle of FIGS. 3 and 4 will be more accepted by a user than display systems which require a user to wear special spectacles.

Since the depth map and the texturing subsequent thereto, as described above, are successively completed gradually, a nearly complete model of the treatment space 2a is provided after some time, which also contains items of information about regions which are presently not visible and are shaded. It is therefore also possible for the image data generator 40 to generate image data from an observation angle which does not correspond to the present position of the sensor device 10. Therefore, for example, a depiction of the treatment space 2a can also be displayed on the visualization device 50, which deviates more or less strongly from the present position of the sensor device 10 and also the surgical instruments 11 optionally arranged on the end of the endoscope. After the depth map has been completed sufficiently, the user can specify the desired viewing direction nearly arbitrarily. In particular by way of the combination of the three-dimensional items of information from the depth map with the further image data of the endoscopic camera and additional items of diagnostic image information, a depiction, which is very close to a depiction of an opened body, can therefore be displayed to a user on the visualization device 50.

For better orientation during the surgical intervention, the user can therefore specify and change the viewing direction arbitrarily according to his or her wishes. This is helpful in particular, for example, if a specific point is to be found on an organ to be treated, or an orientation on the corresponding organ is to be assisted by way of the identification of specific blood vessels or the like.

The specification of the desired viewing direction can be performed in this case by a suitable input device 41. This input device 41 can be, for example, a keyboard, a computer mouse, a joystick, a trackball, or the like. Since the user normally has to operate the endoscope and the surgical instruments 11 contained thereon using both hands during the surgical intervention, however, in many cases, he or she will not have a hand free to operate the input device 41 to control the viewing direction to be specified. The control of the viewing direction can therefore also be performed in a contactless manner in an embodiment. For example, the control of the viewing direction can be carried out via a speech control. In addition, a control of the viewing direction by special, predefined movements is also possible. For example, the user can control the desired viewing direction by executing specific gestures. In particular, it is conceivable that the eye movements of the user are monitored and analyzed. Based on the acquired eye movements, the viewing direction is thereupon adapted for the stereoscopic depiction. Monitoring other body parts of the user to control the viewing direction is also possible, however. Such movements or gestures of the user may be monitored and analyzed by a camera. Alternatively, in the case of a speech control, the input device 41 can be a microphone. However, further possibilities for controlling the predefined viewing direction are also conceivable, for example, by movement of a foot or the like.

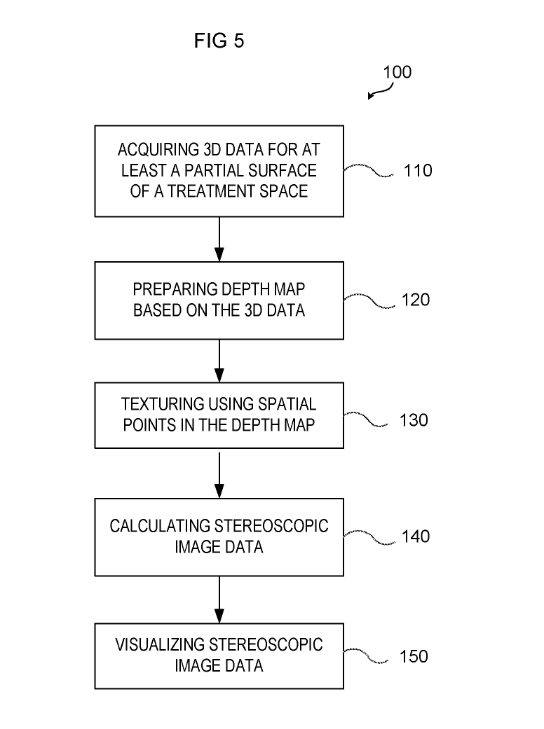

FIG. 5 shows a schematic illustration of a method 100 for the stereoscopic depiction of image data. In 110, firstly a surface of a treatment space 2a is at least partially three-dimensionally acquired. As described above, this three-dimensional acquisition of the surface of the treatment space 2a can be performed by any arbitrary suitable sensor 10. Furthermore, in 120, a depth map is prepared based on the three-dimensional acquisition of the object surface. This prepared depth map contains spatial points of the three-dimensionally acquired surface. Since the sensor device 10 only has a limited viewing angle and additionally partial regions possibly cannot be initially acquired due to shadows, the depth map thus prepared can initially be incomplete at the beginning. By moving the endoscope and therefore also the sensor device 10 inside the treatment space 2a, further spatial points of the surface can be three-dimensionally acquired continuously and these items of information can also be integrated into the depth map. In the event of changes on the acquired surface, corresponding items of information can also be corrected in the depth map.

After a depth map has been prepared using an at least partially three-dimensionally acquired surface, texturing is carried out in 130 using the spatial points present in the depth map. Optionally provided further image data from a camera of the sensor device 10 and/or further items of diagnostic image information from imaging methods such as computer tomography, magnetic resonance tomography, sonography, or x-rays can also be integrated in this texturing. In this manner, initially a three-dimensional colored or black-and-white image of the surface of the treatment space 2a results. Stereoscopic image data are then calculated from the depth map thus textured in 140. These stereoscopic image data include at least two depictions from a predefined viewing direction, wherein the depiction differs in accordance with the eye spacing of an observer. Finally, in 150, the previously calculated stereoscopic image data are visualized on a suitable display device.

The viewing direction, on which the calculation of the stereoscopic image data in 140 is based, can be adapted arbitrarily in this case. In particular, the viewing direction for the calculation of the stereoscopic image data can be different from the viewing direction of the sensor device 10. To set the viewing direction on which the calculation of the stereoscopic image data in 140 is based, the method can include acquiring user input and adapting the viewing direction thereupon for the calculation of the stereoscopic image data in accordance with the user input. The user input for adapting the viewing direction may be performed in a contactless manner in this case. For example, the user input can be performed by analyzing a predefined user gesture.

In summary, a method for the stereoscopic depiction of image data, in particular for the three-dimensional depiction of items of image information during minimally invasive surgery is carried out using an endoscope. In this case, firstly the operation region of the endoscope is three-dimensionally acquired by a sensor device. Stereoscopic image data are generated from the 3D data obtained by sensors and visualized on a suitable display device.

A description has been provided with particular reference to preferred embodiments thereof and examples, but it will be understood that variations and modifications can be effected within the spirit and scope of the claims which may include the phrase “at least one of A, B and C” as an alternative expression that means one or more of A, B and C may be used, contrary to the holding in Superguide v. DIRECTV, 358 F3d 870, 69 USPQ2d 1865 (Fed. Cir. 2004).

1-15. (canceled)

16. A method for stereoscopic depiction of image data in minimally invasive surgery, comprising:
acquiring three-dimensional data representing an at least partial surface;
preparing a depth map of the three-dimensional data;
texturing the depth map to obtain a textured depth map;
calculating stereoscopic image data from the textured depth map;
providing further image information, including at least one of diagnostic image data from computer tomography, magnetic resonance tomography, an x-ray picture, and sonography; and
combining the further image information with the stereoscopic image data.

17. The method as claimed in claim 16, wherein the stereoscopic image data include two viewing directions of two eyes of a user.

18. The method as claimed in claim 16, wherein the depth map includes spatial points of the at least partial surface.

19. The method as claimed in claim 16,
wherein said acquiring of the three-dimensional data is executed continuously; and
wherein said preparing of the depth map is based on said acquiring of the three-dimensional data executed continuously.

20. The method as claimed in claim 16, wherein said calculating calculates the stereoscopic image data for a predefined viewing direction.

21. The method as claimed in claim 20,
further comprising acquiring a user input, and
wherein the predefined viewing direction is adapted based on the user input.

22. A device for the stereoscopic depiction of image data in minimally invasive surgery, comprising
a sensor device acquiring three-dimensional data representing an at least partial surface;
a device preparing a depth map from the three-dimensional data;
a texturing device generating a textured depth map from the depth map;
an image data generator calculating stereoscopic image data from the textured depth map; and
a visualization device visualizing the stereoscopic image data and image information including at least one of diagnostic image data from computer tomography, magnetic resonance tomography, an x-ray picture, and sonography.

23. The device as claimed in claim 22, wherein the sensor device is arranged in an endoscope.

24. The device as claimed in claim 23, wherein the endoscope includes at least one surgical instrument.

25. The device as claimed in claim 22, wherein the sensor device comprises at least one of a time-of-flight camera and a triangulation device performing active triangulation.

26. The device as claimed in claim 22, wherein the image data generator calculates the stereoscopic image data for a predefined viewing direction.

27. The device as claimed in claim 22,
further comprising an input device acquiring an input of a user; and
wherein the image data generator calculates the stereoscopic image data for a viewing direction based on the input of the user.

28. The device as claimed in claim 27, wherein the input device acquires a movement of the user.

29. The device as claimed in claim 27, wherein the input device acquires a gesture of the user.
